(19) United States
(12) Patent Application Publication
(10) Pub. No.: US 2021/0060299 A1
(43) Pub. Date: Mar. 4, 2021

(54) Incisional Tunneler

(71) Applicant: Avent, Inc., Alpharetta, GA (US)

(72) Inventors: Kunal M. Amin, La Mirada, CA (US); Angela M. Mulcahy, South Lyon, MI (US)

(21) Appl. No.: 16/957,770

(22) PCT Filed: Dec. 28, 2017
(86) PCT No.: PCT/US2017/068722

§ 371 Date: Jun. 25, 2020

(51) Int. Cl. A61M 25/01; A61M 25/00; A61M 25/09; A61M 25/06

(52) U.S. Cl. CPC A61M 25/0194; A61M 25/0052; A61M 2202/048; A61M 25/09; A61M 25/0606; A61M 25/0097

(57) ABSTRACT

An incisional tunneler nerve block catheter assembly which includes a catheter and a tunneler, guidewire, or needle contained within the lumen of the catheter. The catheter and the tunneler, guidewire, or needle are configured such that the catheter and the tunneler, guidewire, or needle are inserted simultaneously and are then positioned to a target area. The tunneler, guidewire, or needle are releasably contained within the lumen of the catheter such that a tunneler, guidewire, or needle can be withdrawn from the lumen after insertion or placement such that a drug or anesthesia can be administered through at least one aperture.

### BACKGROUND

Prior to, during, or after performing a surgical operation on a part of the body, it may be desirable to perform a nerve block to anesthetize the nerves in a part of the body proximate to where surgery and incision occur. Often, a catheter-based infusion system, placed with a blunt tunneler/sheath, is utilized to both block the nerve bundles for a surgery or after a trauma, and to provide a continuous, low flow rate of the anesthetic over a period of time (e.g., 2-5 days following surgery) for post-operative pain management.

One approach is to introduce a blunt tunneler and a peel-away-type sheath into the general area of the desired nerves to be blocked. Once proper location of the tunneler/sheath is achieved, the tunneler is removed and a catheter may be introduced through the sheath to administer the anesthetic and maintain the nerve blockade.

Unfortunately, these approaches have several problems. The peel-away sheath and the tunneler both require a second step of feeding the catheter through the sheath after placement. Threading of the catheter through the sheath increases procedure time, and it also requires both hands to properly thread the catheter and remove the sheath. Similarly, as the catheter is inserted after placement, an additional problem with this approach is that the catheter insertion through the sheath maybe difficult.

Therefore, an improved catheter assembly which allows for insertion of a catheter in a single step would be beneficial. Additionally, a catheter assembly where a catheter is introduced simultaneously with a tunneler would be advantageous. Further, it would solve a problem to have a catheter overlie a tunneler that may be removed after the catheter assembly has been placed. An improved catheter assembly may also decrease leakage at the insertion site by providing a catheter larger than the tunneler.

### SUMMARY

In general, the present disclosure is directed to a method for placing a catheter assembly at a target area for a drug delivery. The method may comprise inserting the catheter assembly into an incision, wherein the catheter assembly comprises a catheter extending in an axial direction and having a proximal end and a distal end that define a body that extends from the proximal end to the distal end. The body may have at least one aperture and defines a lumen which extends from the proximal end to the distal end. The distal end may be closed and may have a generally rounded shape. The distal end may be formed from a reinforced material.

The catheter assembly may further comprise a tunneler, guidewire, or needle which is releasably contained within the catheter lumen and wherein the tunneler, guidewire, or needle has a diameter which is smaller than a diameter of the catheter lumen. The catheter assembly may have a hub which may be fixedly or releasably attached to the proximal end of the catheter. The catheter assembly may be placed into the target area, and the tunneler, guidewire, or needle may be removed while the catheter remains in the target area.

Another embodiment of the present disclosure may include a method wherein the catheter is removed after the tunneler, guidewire, or needle has been removed and after a drug or anesthesia has been delivered. In yet a further embodiment, the positioning of the catheter may be adjusted after the tunneler, guidewire, or needle has been removed. Additionally or alternatively, the method of the present disclosure may include providing a continuous flow of a drug or anesthesia to a target area from an opening or aperture in the catheter.

In yet a further embodiment of the present disclosure, the method may include inserting the catheter into an incision, either fully or in part, and then inserting a tunneler, guidewire, or needle into the lumen. Alternatively, the tunneler, guidewire, or needle may be re-inserted into the lumen after the tunneler or guidewire have been removed.

The method of the present disclosure may optionally include bending the catheter into a shape that generally corresponds with a shape of the tunneler, guidewire, or needle prior to insertion. Further, the hub may be secured such that the hub may be used to guide the catheter assembly and position the catheter assembly at the target area.

The present disclosure may also generally be directed to a catheter assembly. The catheter assembly may comprise a catheter having a proximal end and a distal end and a body that extends from the proximal end to the distal end. The body may have at least one aperture and may define a lumen which extends from the proximal end to the distal end. The distal end may be closed and may have a generally rounded shape. At least the distal end of the catheter may be formed from a reinforced material. The catheter assembly may also include a tunneler, guidewire, or needle which may be releasably contained within the lumen such that the tunneler or guidewire have a diameter which is smaller than a diameter of the lumen. The catheter assembly may comprise a hub which may be fixedly or releasably attached to the proximal end of the catheter.

In a further embodiment, a catheter of a catheter assembly of the present disclosure may comprises a polyester, a polyurethane, a polyamide, a polyolefin, or combinations thereof. In yet an additional embodiment, the distal end may be comprised one or more polymers, wherein the polymer comprises a polyester, a polyurethane, a polyamide, a polyolefin, or combinations thereof, wherein the polymer may be hardened so as to have a higher durometer than the base polymer or wherein the polymer has a metal or metal alloy embedded therein. In still yet a further embodiment, the tunneler, guidewire, or needle may be comprised of a metal or metal alloy that is resistant to corrosion and/or oxidation, and/or the hub may be comprised of a rigid plastic such as a polyethylene polymer or copolymer or a polypropylene polymer or copolymer.

The catheter assembly may also include between one and ten apertures. In an additional embodiment, the apertures may be spaced radially, axially, or both radially and axially along the catheter. In an embodiment which includes apertures, the apertures may each have a diameter of about 100 micrometers to about 1 millimeter (mm). Additionally or alternatively, at least a portion of the apertures are located within about 50 mm of the distal end of the catheter. In yet a further embodiment, all of the apertures may be locate within about 30 mm of the distal end of the catheter.

In another embodiment, the catheter of the catheter assembly may have a diameter of about 4 mm to about 8 mm. In a further embodiment, the catheter may have a diameter of about 6 mm.

### BRIEF DESCRIPTION OF THE DRAWINGS

The invention will now be described, by way of example, with reference to the accompanying drawings, in which:

FIG. 1A illustrates a side perspective view of the catheter assembly of an embodiment according to the present disclosure;

FIG. 1B illustrates a side perspective view of the catheter assembly of FIG. 1A showing the embodiment with a partially withdrawn tunneler, guidewire, or needle;

FIG. 1C illustrates a side perspective view of the catheter assembly of FIG. 1A showing the embodiment with the tunneler, guidewire, or needle fully withdrawn;

FIG. 2 illustrates a side perspective view of the catheter assembly of the present disclosure showing the embodiment wherein a tunneler, guidewire, or needle is being inserted or reinserted into the lumen;

FIG. 3 illustrates a close up view of the distal end of an embodiment of the present disclosure;

FIGS. 4A and 4B illustrate cross-sectional views of embodiments of the catheter assembly according to the present disclosure; and

FIGS. 5A and 5B illustrate cross-sectional views of embodiments of the catheter according to the present disclosure.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

The positional terms “proximal” and “distal” are used herein to orient the various components relative to each other. “Distal” refers to the direction that would be closest to a catheter insertion site in a patient, and “proximal” refers to the opposite direction.

Generally, the present disclosure is directed to a catheter assembly wherein a tunneler, guidewire, or needle is releasably disposed with a lumen of a catheter as well as a method of using the catheter assembly. Such an arrangement may allow a catheter and a tunneler, guidewire, or needle of a catheter assembly to be inserted and placed simultaneously. Additionally or alternatively, the catheter assembly of the present disclosure may comprise a catheter which has a distal end which may be generally rounded and be comprised of a reinforced material. By using a durable material or reinforced material on at least a distal tip of the catheter, the catheter may be used over a tunneler, where the catheter is able to withstand the forces applied during placement of the catheter assembly. The catheter assembly of the present disclosure may also comprise a hub releasably or fixedly attached to a proximal end of the catheter. When used, a hub may be shaped and comprised of a material such that the hub may be used to position the catheter assembly and withstand the forces being applied thereto. A catheter assembly of the present disclosure may further include at least one aperture such that the catheter assembly may be used to deliver a drug or anesthesia to a target area.

A benefit of the catheter assembly of the present disclosure is that the insertion of the catheter for an incisional nerve block may be conducted in a single step. This allows for time savings and also may provide the benefits of easier placement of a catheter in a target area as well as more accurate placement of a catheter in a target area. Additionally, the catheter assembly of the present disclosure may utilize a catheter which would allow repositioning of the catheter after removal of the tunneler, guidewire, or needle, or alternatively or additionally, re-insertion of the catheter and a tunneler, guidewire, or needle to allow for repositioning.

Accordingly, the catheter assembly generally includes a catheter having a proximal end and a distal end and a body that extends in an axial direction A from the proximal end to the distal end. The distance between the proximal and distal ends of the catheter may generally correspond to the length of a tunneler, guidewire, or needle used with the catheter, or the catheter may have a greater or lesser length than a tunneler, guidewire, or needle. For instance, the length of the catheter, tunneler, guidewire, or needle may be greater than about 2 centimeters, such as about 5 centimeters or greater, such as about 7 centimeters or greater, such as about 10 centimeters or greater, such as about 12 centimeters or greater, such as about 15 centimeters or greater, such as about 20 centimeters or greater, such as about 25 centimeters or greater, such as about 30 centimeters or greater, such as about 35 centimeters or greater, such as about 40 centimeters or greater, such as about 45 centimeters or greater, such as about 50 centimeters or greater, such as about 55 centimeters or greater, such as about 60 centimeters or greater, such as less than about 80 centimeters, such as about 75 centimeters or less, such as about 70 centimeters or less, such as about 65 centimeters or less, such as about 60 centimeters or less, such as about 55 centimeters or less, such as about 50 centimeters or less, such as about 45 centimeters or less, such as about 40 centimeters or less, such as about 35 centimeters or less, such as about 30 centimeters or less, such as about 25 centimeters or less, such as about 20 centimeters or less, such as about 15 centimeters or less in length. The catheter may include a piezoelectric or echogenic component as known in the art that may be configured within the body material or inside of the catheter, or alternatively may include either no positioning aid, or another positioning aid known in the art.

The catheter may generally have a shape and size as generally known in the art such as described by the French gauge scale. Alternatively, the catheter may be tapered towards the distal end. In one embodiment, the catheter may be tapered such that the lumen of the catheter is also tapered, or alternatively only the exterior of the body is tapered such that the diameter of the catheter is tapered without changing the diameter of the lumen. The lumen may have a length and diameter generally corresponding to the diameter and length of a tunneler, guidewire, or needle that may be used with the catheter, or may have a smaller or larger length or larger diameter than a tunneler, guidewire, or needle that may be used with the catheter. Regardless of the length and/or outer diameter of the catheter, the lumen may have a diameter larger than a diameter of a needle used with the catheter assembly of the present disclosure. In one embodiment, a catheter may have an outer diameter of at least about 2 mm, such as at least about 4 mm, such as at least about 5 mm, such as at least about 6 mm, such as at least about 7 mm, such as at least about 8 mm, such as less than about 12 mm, such as less than about 11 mm, such as less than about 10 mm, such as less than about 9 mm, such as less than about 8 mm. Additionally, the catheter body may have a thickness t which is defined as the distance between the outer diameter of the catheter and the inner diameter of the catheter or the lumen diameter.

The catheter body of the present disclosure may also generally have a rounded distal end. Such a configuration may allow the catheter assembly to be placed in a target area while causing less damage to nerves or tissues than a sharp or pointed introducer, tunneler, or needle. A rounded distal end of the present disclosure may be generally comprised of the same or similar material as the catheter body. Additionally or alternatively, the material be a different material than the catheter or may be the same material as the catheter body which further comprises a reinforcing material. A reinforcing material or material for forming the distal tip may generally include various materials including polyesters, polyurethanes, polyamides, polyolefins, including polyethylene and polypropylene, and any copolymers thereof, such as nylon, polyester elastomer, polyether/block polyamide, tetrofluorethylenes or in one embodiment, a substituted or unsubstituted, polyamide polymer, such as nylon, that may undergo additional reinforcement, such as by hardening or increasing the durometer of the nylon, or by embedding a metal or metal alloy therein. By using a reinforcing material, the distal tip may be able to withstand the pressure applied to the catheter assembly during insertion such that a catheter may overlie a tunneler, guidewire, or needle and help to eliminate the need for a separate introducer, tunneler, guidewire, or needle.

The body of the catheter may be comprised of various materials including polyesters, polyurethanes, polyamides, polyolefins, including polyethylene and polypropylene, and any copolymers thereof, such as nylon, polyester elastomer, polyether/block polyamide, and/or tetrofluorethylene. Moreover, the body of the catheter has a thickness, wherein the thickness of the body is defined as the distance between the outer diameter of the catheter body and the inner diameter of the catheter or the diameter of the catheter lumen and as generally shown by reference character t. For instance, the thickness of the body t may be greater than about 500 micrometers, such as about 600 micrometers or greater, such as about 700 micrometers or greater, such as about 800 micrometers or greater, such as about 900 micrometers or greater, such as about 1000 micrometers or greater, such as about 1100 micrometers or greater, such as about 1200 micrometers or greater, such as about 1300 micrometers or greater, such as about 1600 micrometers or less, such as about 1500 micrometers or less, such as about 1400 micrometers or less, such as about 1300 micrometers or less, such as about 1200 micrometers or less, such as about 1100 micrometers or less, such as a thickness of about 1000 micrometers or less. The body material and thickness may be selected based upon the target location of the catheter, the likelihood of positioning the catheter after removal of the needle, and additional considerations. For example, a thicker body or a stronger material may be selected if the catheter may need to be repositioned after removal of the needle or a thinner or weaker material may be selected so as to decrease bulk and potentially increase comfort in areas where the catheter is less likely to need to be repositioned. It should also be noted that a reinforcing material may be used alone or in combination with a thicker or stronger material in situations where no needle, tunneler, or guidewire is needed, and instead the catheter may be used without the tunneler, needle, or guidewire. A reinforcing material or a thicker or stronger body may also allow the catheter of the present disclosure to be used independently, as compared to prior catheters which required the use of a sleeve or additional overlying materials in order to withstand the forces applied during placement.

The catheter body may further comprise at least one aperture. While there may be more or less apertures, the catheter body may generally contain at least 1 aperture, such as at least about 2, such as at least about 3, such as at least about 4, such as at least about 5, such as at least about 7 such as at least about 10 apertures. The catheter body may also comprise less than about 20, such as less than about 18, such as less than about 15, such as less than about 13 apertures, such as less than about 10 apertures.

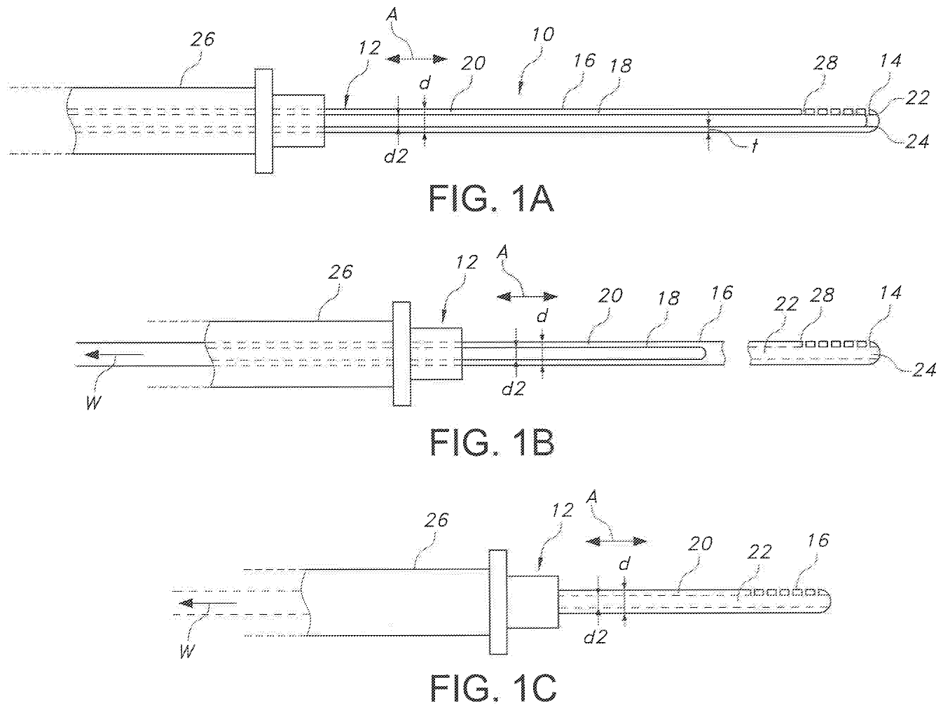

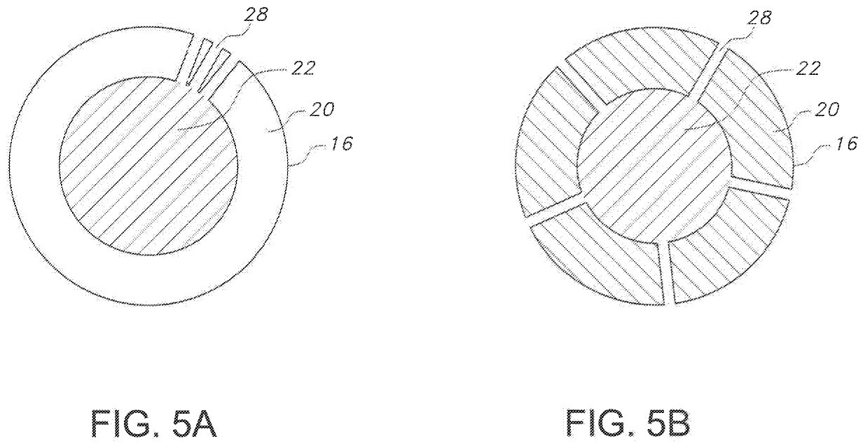

The apertures may be spaced apart radially as generally shown in FIG. 5, axially as generally shown in FIGS. 1A-1C, or both radially and axially along the catheter body. In one embodiment, the apertures may be generally located adjacent to the distal end, such as within about 50 mm from the distal end, such as within about 45 mm, such as within about 40 mm, such as within about 35 mm, such as within about 30 mm, such as within about 25 mm, such as within about 20 mm, such as within about 15 mm, such as within about 10 mm of the distal end. In another embodiment, the apertures may be evenly or randomly spaced radially, axially, or a combination of radially and axially along the entirety of the length of the catheter body.

The apertures may have a diameter of about 1 millimeter or less, such as about 900 micrometers or less, such as about 800 micrometers or less, such as about 700 micrometers or less, such as about 600 micrometers or less, such as about 500 micrometers or less, such as about 400 micrometers or less, such as about 300 micrometers or less, such as about 100 micrometers or greater, such as about 200 micrometers or greater, such as about 300 micrometers or greater, such as about 400 micrometers or greater, such as about 500 micrometers or greater, such as about 600 micrometers or greater in diameter. In a further embodiment, some of the apertures may have a fairly large diameter and some of the apertures may have a fairly small diameter. In an embodiment with apertures that have a mixture of diameters, the ratio of the diameter types may be fairly even, or in an alternate embodiment there may be more large diameter apertures, such as twice as many large, such as three times as many large, such as five times as many large diameter apertures. Alternatively, there may be more small diameter apertures such as twice as many small, such as three times as many small, such as five times as many small diameter apertures.

The arrangement of the apertures, number of apertures, and diameter of the apertures may be selected based upon the target area, the type of drug or anesthesia to be delivered, or the desired rate of delivery, to name a few considerations. For example, a larger number of apertures or apertures with a larger diameter, or both, may be selected when a greater rate of delivery is desired. Alternatively, arrangements of apertures may be selected based upon the location and orientation of the target area. For example, if delivery of a drug to a small target area is desired, all of the apertures may be located fairly close together either radially and/or axially. In an additional embodiment for example, if a target area is located on one side of a catheter, then the apertures may mainly be located on a single side of the catheter body such that the apertures are all located on generally the same side, area, or radius. Alternatively, if a more central location is chosen for placement of the catheter, the apertures may extend radially around the catheter body such that an aperture is located on several different radii around the catheter body.

The catheter assembly of the present disclosure may also generally include a tunneler, guidewire, or needle. The tunneler, guidewire, or needle may have a shape and size that may be selected based upon the target area or based upon the size of catheter desired. In a further embodiment, a curved tunneler, guidewire, or needle may be used, or the tunneler, guidewire, or needle may be bent to form a shape, and in such a case a catheter formed from a flexible material capable of holding a shape may be used so as to maintain the shape after a tunneler, guidewire, or needle is removed from the catheter assembly. A tunneler, guidewire, or needle of the present disclosure may also be made of an echogenic material, or include other materials based upon the target area and placement thereof. The tunneler, guidewire, or needle may be selected to correspond with a size and shape of a catheter lumen of the present disclosure, or a standard sized and shaped needle may be used.

Regardless of the tunneler, guidewire, or needle selected, the diameter of the tunneler, guidewire, or needle may be chosen so as to substantially occupy the lumen of the catheter or may occupy the entirety of the lumen. Such an embodiment may allow greater stability during placement and may impart greater durability and strength to the catheter assembly. In an alternative embodiment, the tunneler, guidewire, or needle may occupy less than the entirety of the lumen. Such an embodiment may be used wherein a drug or anesthesia may be delivered while the tunneler, guidewire, or needle is still partially or completely inserted in the lumen. An embodiment wherein the tunneler, guidewire, or needle occupies less than all of the lumen may be selected when the catheter assembly may need to be repositioned, however, it should be noted that a catheter of the present disclosure may not need a tunneler, guidewire, or needle in order to be repositioned.

The needle, tunneler, or guidewire may generally have a length or be inserted into the catheter lumen at a location so as to provide structural strength, guideability, and positionability to the catheter assembly. In such an embodiment, the tunneler, guidewire, or needle may be comprised of a metal or metal alloy that may be resistant to corrosion and/or oxidation, in a further embodiment, the metal or metal alloy may be an iron based alloy such as stainless steel. In one embodiment the tunneler, guidewire, or needle may be used initially, and after which it can be withdrawn from the catheter assembly, such that placement may be completed after withdrawal of the tunneler, guidewire, or needle. The tunneler, guidewire, or needle may also be configured so as to remain in the lumen in a retracted position wherein the tunneler, guidewire, or needle still allows the delivery of a drug or anesthesia.

In yet a further embodiment, the catheter assembly may also include a hub releasably or permanently attached or configured at the proximal end of the catheter. The present disclosure may include both a tunneler, guidewire, or needle hub and a catheter hub, or may include a singular hub suitable to interact with both a tunneler, guidewire, or needle and a catheter. Regardless of the type and number of hubs, the hub or hubs may be configured to maintain the needle, tunneler, or guidewire within the lumen of the catheter until it is desired to remove the tunneler, guidewire, or needle. Alternatively, the hub may comprise a durable material so as to not become disconnected from the catheter and tunneler, guidewire, or needle when the catheter assembly is placed within a target area, such as a rigid plastic such as a polyethylene polymer or copolymer or a polypropylene polymer or copolymer. In yet a further embodiment, the hub or hubs may provide a convenient place to hold and/or guide the catheter assembly. Additionally or alternatively, the hub may be provided with ports for administering a drug or anesthesia through the catheter, or a tunneler, guidewire, or needle, or both, or may provide a connection to the needle or catheter such as a nerve stimulator device.

The present disclosure may further be generally directed to a method for administering a bolus or continuous flow of a drug or anesthetic to a patient. The catheter assembly may generally have the form of the catheter assembly as discussed above. The components of the catheter assembly may be inserted into an incision simultaneously and placed at a target area. The catheter assembly may be fully positioned before withdrawing or removing the tunneler, guidewire, or needle, or, alternatively, the catheter assembly may only be partially inserted before withdrawing or removing the tunneler, guidewire, or needle, and may then be further positioned after withdrawing or removing the tunneler, guidewire, or needle.

The catheter assembly may further be used to administer a drug or anesthesia to a patient or a target area of a patient. The target area may be a nerve or nerve bundle, or alternatively may be used for other or more general targets. Administration of the drug or anesthesia may begin while the tunneler, guidewire, or needle is still contained within the catheter lumen or may not begin until the tunneler, guidewire, or needle has been either withdrawn into the lumen or withdrawn from the lumen. As previously discussed, the shape and size of the catheter, and the shape, size and orientation of the apertures may be selected based upon the amount or type of drug or anesthesia, or based upon the desired rate of delivery, to name a few considerations.

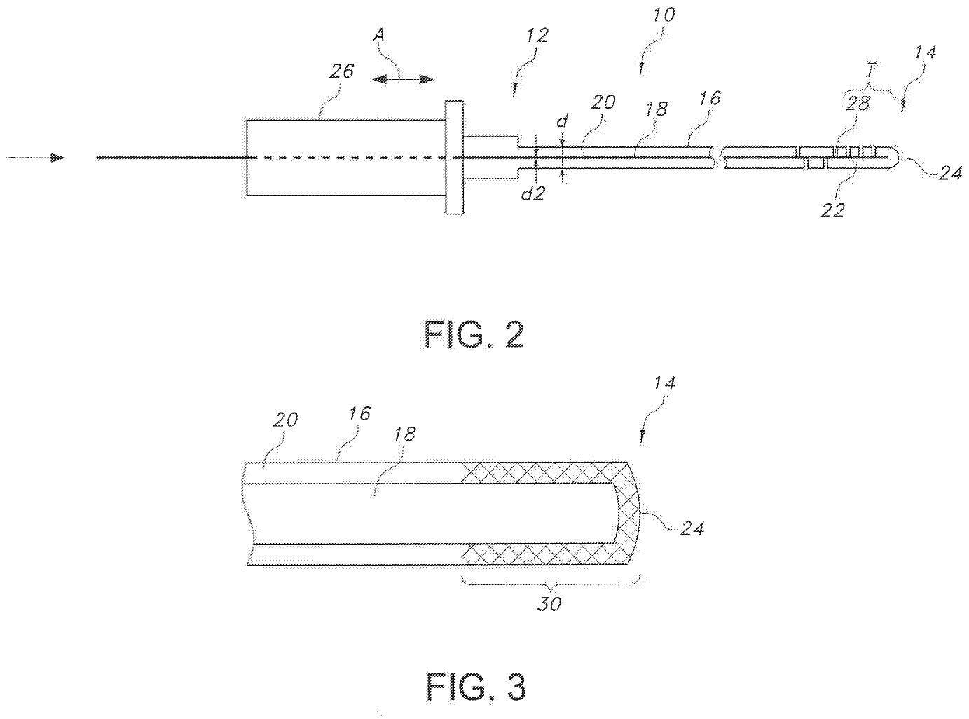

While embodiments of the present disclosure have been generally discussed, various embodiments of the present disclosure will now be described in more detail. Referring now to the drawings, FIGS. 1A-5B illustrate various embodiments of the catheter assembly 10 according to the present disclosure. It should be understood that the catheter assembly of the present disclosure may have any suitable catheter assembly configuration known in the art. For example, in certain embodiments such as FIG. 1A the catheter assembly 10 may have a proximal end 12 and a distal end 14, and a tunneler, guidewire, or needle 18 may be coaxially located in a lumen 22 of a catheter 16 such that catheter 16 and tunneler, guidewire, or needle 18 may be simultaneously inserted into a patient. Further, as shown, the catheter 16 has a catheter body 20 that extends from the proximal end 12 to the distal end 14 in the axial direction A. The catheter body defines a lumen 22 (shown more clearly in FIGS. 2 and 3) in which a tunneler, guidewire, or needle 18 has been inserted thereto. The lumen 22 extends between the catheter body 20 and may have a diameter d2 that is larger than a diameter of a tunneler, guidewire, or needle 18. The catheter body may have a thickness t, which may be generally constant from the proximal end 12 to the distal end 14 of the catheter assembly 10, or which may increase or decrease, wherein the thickness t is defined as the distance between the outer diameter d of the catheter 16 and the inner diameter d2 of the catheter 16 or the lumen diameter d2.

As shown in FIG. 1A, catheter 16 may have a rounded distal tip 24. In addition, the proximal end 12 of the catheter 16 may include a hub 26 configured thereon. The hub 26 may further be configured to interact with both the tunneler, guidewire, or needle 18 and the catheter 16, or the multiple hubs 26 which may be configured to interact with each other may be used such that a hub is used for the catheter and a hub is used for the tunneler, guidewire, or needle. Though not shown, a fluid input or delivery device may be attached to the hub or hubs. It should be understood that any catheter assembly 10 described herein may include at least one aperture 28 for delivery of a drug or anesthesia to a target area or patient.

In FIG. 1A the lumen 22 is shown as occupying a substantial portion of the catheter 16. In an alternative embodiment, the lumen 22 may occupy less of the catheter 16 volume and the catheter body 20 may be thicker than in FIG. 1A, wherein the thickness t is defined as the distance between the outer diameter d of the catheter and the inner diameter d2 of the catheter or the lumen diameter d2. Such an embodiment may provide for a catheter with increased strength or durability, and may utilize a smaller tunneler, guidewire, or needle. Alternatively, a lumen 22 may occupy a substantial volume of catheter 16 and the catheter body 20 may be comprised of a reinforcing material that may provide for increased strength and durability of the catheter 16. In yet a further embodiment, the outer diameter d of the catheter may be similar to that as shown in FIG. 1A, but the thickness t of the catheter body 20 may be increased and the lumen diameter d2 may be decreased such that the outer diameter d of the catheter remains fairly similar to the embodiment of FIG. 1A. In yet a further embodiment, a standard catheter may be used and strength and durability may be imparted by the tunneler, guidewire, or needle.

FIG. 1B generally shows the embodiment of FIG. 1A wherein the tunneler, guidewire, or needle 18 has been partially removed and is withdrawn towards a proximal end 12 of the catheter 16 shown by direction W. The tunneler, guidewire, or needle 18 may remain in this position in an embodiment wherein the catheter 16 and/or the tunneler, guidewire, or needle 18 have been configured to allow for delivery of a drug while the tunneler, guidewire, or needle 18 remains in the lumen 22. Alternatively, the tunneler, guidewire, or needle 18 may be further withdrawn from the lumen 22 in the direction W such as shown in FIG. 1C.

FIG. 2 refers to an embodiment of the present disclosure wherein the tunneler, guidewire, or needle 18 is being initially inserted into the lumen 22, or re-inserted into the lumen 22 after withdrawal from the lumen 22. In FIG. 2, an embodiment is shown wherein a catheter 16 with a smaller outer diameter d is used, and the volume occupied by lumen 22 and catheter body 20 remain proportionally similar to FIGS. 1A-1C in terms of ratio of thickness of the lumen 22 and catheter body 20, such that catheter assembly 10 has a lumen 22 with a smaller diameter d2. However, in an additional embodiment, the ratio of the diameter d2 of the lumen to the diameter d of the catheter body 20 may not remain roughly the same and may increase or decrease. Such an embodiment may be used when a smaller target area is desired or wherein a space to access a target area is narrow, to name a few considerations.

FIG. 3 generally shows a close up of a distal end 14 of the catheter 16 of an embodiment of the present disclosure. As shown, the distal end 14 may have a reinforced section 30. The reinforced section 30 may cover the rounded distal tip 24 and may extend axially along the catheter body 20 toward the proximal end 12. The reinforced section 30 may extend a distance in the axial direction such as about 5 mm from the distal end 14, such as about 10 mm or greater, such as about 15 mm or greater, such as about 20 mm or greater, such as about 25 mm or greater, such as about 30 mm or greater, such as about 35 mm or greater, such as about 40 mm or greater, such as about 45 mm or greater, such as about 50 mm or greater from the distal end, such as bout 250 mm or less, such as about 200 mm or less, such as about 150 mm or less, such as about 100 mm or less, however, in one embodiment, the entirety of the catheter body 20 may be reinforced or alternatively, none of the catheter body 20 may be reinforced. Though not shown, the reinforced section 30 may also include at least one aperture 28. Alternatively, the apertures may only be located on a section of the catheter body 20 that does not have a reinforcing material. In yet a further embodiment, the apertures 28 may be located in both reinforced areas and unreinforced areas. The reinforcing material such as that shown in FIG. 3 provides additional strength and support to the catheter such that it is not necessary to use a tunneler, guidewire, or needle, or an additional sheath. By providing a reinforcing material, particularly at the distal end 14, the catheter body 20 can withstand the forces applied during placement without the need of an additional sheath or sleeve.

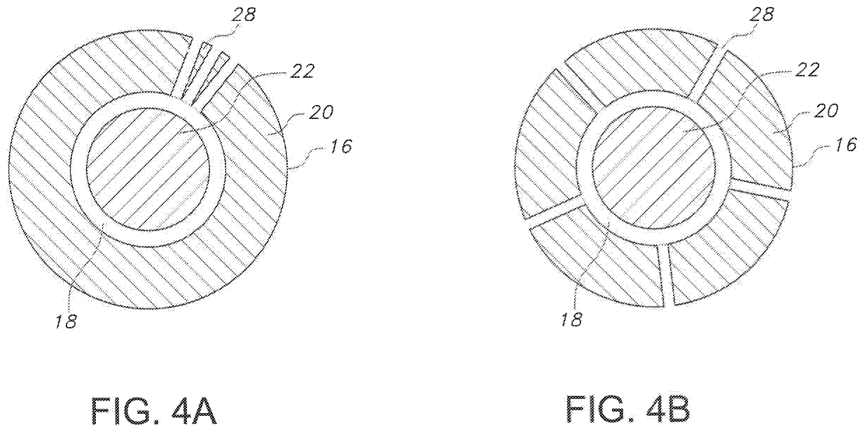

FIGS. 4A and 4B show cross sections taken proximal from the tip of the catheter assembly of the present disclosure. FIGS. 4A-4B show a catheter body 20 defining a lumen 22, wherein a needle 18 is disposed within the catheter lumen 22. As shown, the diameter of the needle 18 is smaller than a diameter of the lumen 22. As shown in FIGS. 4a and 4b, apertures 28 may be disposed axially around the catheter body 20 as shown in 5b or may be located generally in a single axial plane such as shown in FIG. 4A. Alternatively, though not shown in FIGS. 4A and 4B, apertures 28 may be spaced both radially and axially along catheter body 20 as shown generally in FIG. 2.

FIGS. 5A and 5B show cross sections taken proximal from the tip, similar to FIGS. 4A and 4B, except that in the present embodiment, the needle 18 has been withdrawn from this portion of the lumen 22. Apertures 28 may be disposed axially around the catheter body 20 as shown in FIG. 5B or may be located generally in a single axial plane such as shown in FIG. 5A.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. A method for placing a catheter assembly at a target area for drug delivery comprising:
inserting the catheter assembly into an incision,
the catheter assembly comprising: a catheter extending in an axial direction and having a proximal end and a distal end that define a body that extends from the proximal end to the distal end, the body having at least one aperture, and defining a lumen which extends from the proximal end to the distal end, the catheter having a closed distal end with a rounded shape, wherein at least the distal end of the catheter is formed from a reinforced material,
a tunneler, guidewire, or needle which is releasably contained within the catheter lumen such that the tunneler, guidewire, or needle has a diameter which is smaller than a diameter of the catheter lumen; and
a hub, wherein the hub is fixedly or releasably attached to the proximal end of the catheter;
placing the catheter assembly into the target area; and
removing the tunneler, guidewire, or needle while the catheter remains in the target area.

2. The method of claim 1, further comprising a step of removing the catheter after the tunneler, guidewire, or needle has been removed and after a drug has been delivered.

3. The method of claim 1, further comprising a step of adjusting placement of the catheter after the tunneler, guidewire, or needle has been removed.

4. The method of claim 1 further comprising a step of providing a continuous flow of a drug to a target area from an opening or aperture in the catheter.

5. The method of claim 1, wherein the tunneler, guidewire, or needle is inserted into the lumen after the catheter has been fully or partially inserted into the incision, or wherein the tunneler, guidewire, or needle is re-inserted into the lumen after the tunneler, guidewire, or needle has been removed from the catheter.

6. The method of claim 1, further comprising a step of bending the catheter into a shape that corresponds with a shape of the tunneler prior to insertion.

7. The method of claim 1, wherein the hub is fixedly secured to the catheter.

8. The method of claim 7, wherein the hub in secured to the catheter such that the hub may be used to guide the catheter assembly into the target area.

9. A catheter assembly comprising,
a catheter having a proximal end and a distal end and a body that extends from the proximal end to the distal end, the body having at least one aperture, and defining a lumen which extends from the proximal end to the distal end, the catheter having a closed distal end with a rounded shape, and wherein at least the distal end of the catheter is formed from a reinforced material;
a tunneler, guidewire, or needle which is releasably contained within the lumen such that the tunneler, guidewire, or needle has a diameter which is smaller than a diameter of the lumen; and
a hub which is fixedly or releasably attached to the proximal end of the catheter.

10. The catheter assembly of claim 9, wherein the catheter comprises a polyester, a polyurethane, a polyamide, a polyolefin, or a combination thereof.

11. The catheter assembly of claim 9, wherein the distal end comprises a polymer, wherein the polymer comprises a polyester, a polyurethane, a polyamide, a polyolefin, or a combination thereof, wherein the polymer has been hardened or has a metal or metal alloy embedded in the polymer.

12. The catheter assembly of claim 9, wherein the tunneler, guidewire, or needle comprises a metal or metal alloy.

13. The catheter assembly of claim 9, wherein the hub comprises a rigid plastic.

14. The catheter assembly of claim 9, wherein the catheter includes between 1 and 10 apertures for delivery of a the drug.

15. The catheter assembly of claim 14, wherein the apertures are spaced radially, axially, or both radially and axially along the catheter.

16. The catheter assembly of claim 14, wherein each aperture has a diameter of about 100 micrometers to about 1 millimeter.

17. The catheter assembly of claim 14, wherein at least a portion of the apertures are located within about 50 mm of the distal end of the catheter.

18. The catheter assembly of claim 14, wherein the apertures are located within about 30 mm of the distal end of the catheter.

19. The catheter assembly of claim 9, wherein the catheter has a diameter of about 4 mm to about 8 mm.

20. The catheter assembly of claim 9, wherein the catheter has a diameter of about 6 mm.
